(19) United States
(12) Patent Application Publication
(10) Pub. No.: US 2016/0199222 A1
(43) Pub. Date: Jul. 14, 2016

(54) Ice Pack Holder

(71) Applicants: Charles A. Mullen, SR., San Diego, CA (US); Cassandra L. Mullen, San Diego, CA (US)

(72) Inventors: Charles A. Mullen, SR., San Diego, CA (US); Cassandra L. Mullen, San Diego, CA (US)

(21) Appl. No.: 14/988,972

(22) Filed: Jan. 6, 2016

Related U.S. Application Data
(60) Provisional application No. 62/101,015, filed on Jan. 8, 2015.

(51) Int. Cl. A61F 7/02

(52) U.S. Cl. CPC A61F 7/02; A61F 2007/0231; A61F 2007/023

(57) ABSTRACT

An ice pack holder for use in holding ice packs or hot compresses against a portion of a user's body. The ice pack holder includes a frame defining an open central area, wherein netting extends across the frame and over the open central area. The frame and netting are used to hold an ice pack in position on the user's body. One or more securing members extend from the frame for use in securing the frame in position on the user's body. The securing members preferably include straps that can be wrapped around a user's body part and secured thereon via adjustable fasteners. The ice pack holder can be used to apply an ice pack or similar item to any of various portions of the user's body including the user's arms, legs, shoulder, and joints.

### CROSS REFERENCE TO RELATED APPLICATION

This application claims the benefit of U.S. Provisional Application No. 62/101,015 filed on Jan. 8, 2015. The above identified patent application is herein incorporated by reference in its entirety to provide continuity of disclosure.

### BACKGROUND OF THE INVENTION

### Field of the Invention

The present invention relates to holding devices. More specifically, the present invention provides an ice pack holder for holding ice packs or heating packs in a desired position on a user's body.

It is often necessary to apply hot or cold compresses to an injured portion of a person's body in order to soothe the injury and to promote healing and recovery. In order to apply an ice pack or similar device to the injured area, the person must generally hold the ice pack in position on the injured area. This can be inconvenient for the person and the person may become tired from holding the ice pack in the desired position. Alternatively, ice packs and the like are often secured to the injured body part using tape or wraps. However, using tape or wraps may be uncomfortable, and once the ice pack is secured beneath the tape or wrap, it can no longer be adjusted. Thus, an improved device for holding an ice pack in position on a user's body is desired.

Devices have been disclosed in the prior art that relate to devices for supporting ice packs and the like. These include devices that have been patented and published in patent application publications. These devices generally relate to devices for applying thermal therapy, such as cold packs, to a user, such as U.S. Pat. No. 6,589,272, U.S. Pat. No. 7,815,584, U.S. Pat. No. 4,688,572, U.S. Pat. No. 7,481,786, U.S. Pat. No. 7,022,093, and U.S. Published Patent Application Publication 2008/0077214.

These prior art devices have several known drawbacks. The devices in the prior art fail to provide an ice pack holder that can be removably applied to any of various portions of a user's body. Such devices are constructed for application to a specific area, and many devices are not suited for supporting an ice pack or the like in a specific position.

In light of the devices disclosed in the prior art, it is submitted that the present invention substantially diverges in design elements from the prior art and consequently it is clear that there is a need in the art for an improvement to existing ice pack holding devices. In this regard the instant invention substantially fulfills these needs.

### SUMMARY OF THE INVENTION

In view of the foregoing disadvantages inherent in the known types of ice pack holders now present in the prior art, the present invention provides a new ice pack holder wherein the same can be utilized for providing convenience for the user when applying hot or cold compresses to an injured portion of the user's body. The ice pack holder comprises a frame having a netting thereon, wherein the frame and netting are used to secure an ice pack in position on a user's body. The frame includes one or more securing members securely fastening the ice pack holder in position on a portion of a user's body. The securing members include adjustable fasteners thereon such as hook and loop fastening material, so as to allow the tightness to be adjusted.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front view of an embodiment of the ice pack holder.

FIG. 2 shows a front view of an alternate embodiment of the ice pack holder.

FIG. 3 shows a perspective view of the ice pack holder in use.

### DETAILED DESCRIPTION OF THE INVENTION

Reference is made herein to the attached drawings. Like reference numerals are used throughout the drawings to depict like or similar elements of the ice pack holder. For the purposes of presenting a brief and clear description of the present invention, the preferred embodiment will be discussed as used for holding an ice pack in position on a user's body. The figures are intended for representative purposes only and should not be considered to be limiting in any respect.

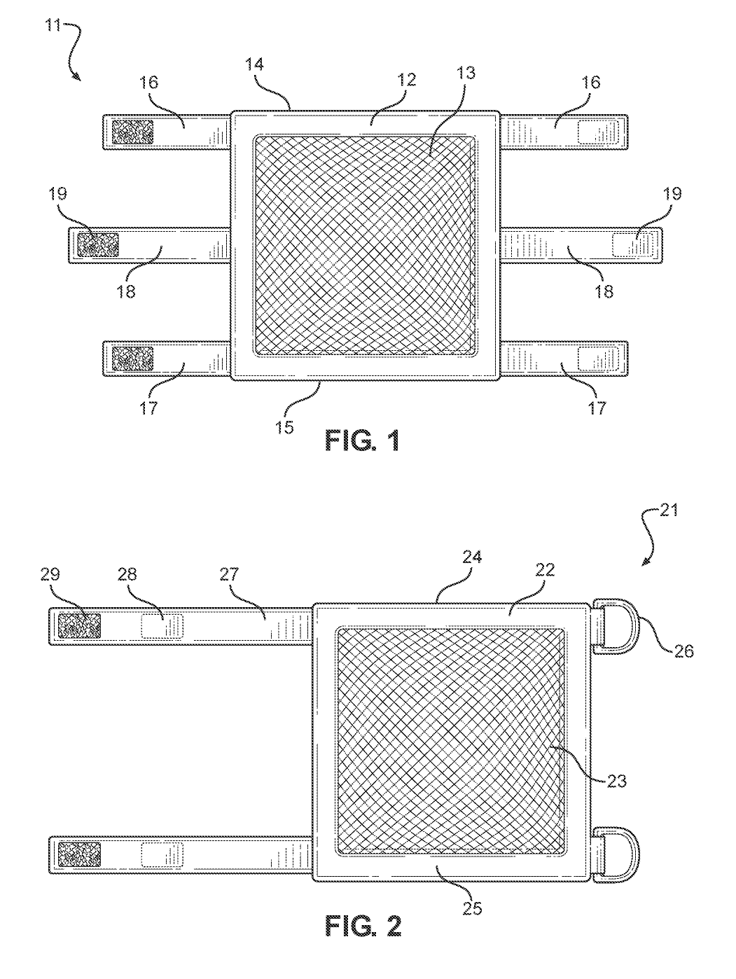

Referring now to FIG. 1, there is shown a front view of an embodiment of the ice pack holder. The ice pack holder 11 comprises a frame 12 defining an open central area. A netting 13 extends between the frame 12 and across the open central area. The frame 12 is preferably composed of a flexible material so as to fit along the contours of a user's body, and in some embodiments the frame 12 is composed of a fabric or nylon material. The netting 13 may be composed of a stretchable or elastic material so as to apply tension to the ice pack or heated compress held thereunder in order to hold the ice pack in the desired position.

The frame 12 includes an upper end 14 and a lower end 15. One or more securing members are disposed on the frame 12 and are used to affix the frame 12 to a portion of the user's body, such as an arm or leg, among others. In the illustrated embodiment, the securing members comprise straps 16, 17, 18 that extend from opposing sides of the frame 12. The straps 16, 17, 18 are adapted to be wrapped around a body part of a user and secured therearound so as to hold the ice pack holder in a desired position on the user's body. In the illustrated embodiment, a pair of upper straps 16 extends from opposing sides of the upper end 14 of the frame 12. A pair of lower straps 17 extends from opposing sides of the lower end 15 of the frame 12. Further, a pair of center straps 18 extend from opposing sides of a central portion of the frame 12.

The straps 16, 17, 18 are preferably adjustably securable around the body of the user so as to provide a desired tightness or fit. The straps 16, 17, 18 include an adjustable fastener 19 thereon. In the illustrated embodiment, the adjustable fastener 19 comprises hook and loop fastening material disposed on each strap. Each pair of straps 16, 17, 18 can be secured to one another around a user's body part by securing the hook and loop fasteners thereon in an overlapping configuration. In alternate embodiments other adjustable fasteners are used, such as buckles and the like.

Referring now to FIG. 2, there is shown a front view of an alternate embodiment of the ice pack holder. The ice pack holder 21 comprises a frame 22 having an upper end 24, a lower end 25, and an open central area. The open central area is covered by a netting 23 that extends across the frame 22. In the illustrated embodiment, the ice pack holder 21 comprises an alternate type of securing member. A first side of the frame 22 includes one or more D-rings 26 thereon. The second side of the frame 22, opposite the first side includes one or more straps 27 extending therefrom. In operation, the user can position the frame 22 on the desired position of his or her body. The user can then wrap the strap 27 around his or her body and insert the strap through the D-ring 26 on the opposing side of the frame 22 so as to encircle a part of the user's body. The strap 27 includes mating fasteners 28, 29 thereon that can be secured in an overlapping configuration in order to secure the ice pack holder 22 on the user's body.

The frame 22 is provided in any of various shapes and sizes depending upon the area on which the frame 22 is adapted to be positioned. Preferably, the frame 22 is shaped so as to fit over a specific part of the user's body. Thus, the frame 22 may be suited for positioning over the user's knee. In alternate embodiments, the frame 22 is suited for positioning on a user's shoulder.

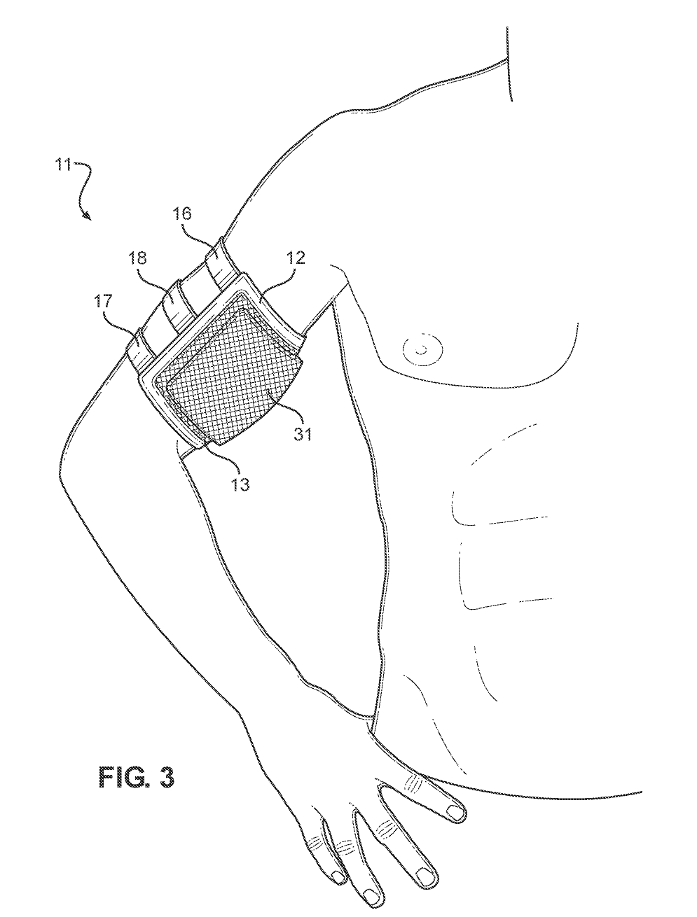

Referring now to FIG. 3, there is shown a perspective view of the ice pack holder in use. In operation, the user can position an ice pack 31 in a desired position on the user's body, such as on the user's arm. The user can then position the netting 13 over the ice pack 31 so as to hold the ice pack 31 in position on the user's body part. The frame 12 is composed of a flexible material so as to contour to and fit the shape of the user's body part. The frame 12 and netting 13 help to secure the ice pack 31 in the desired position.

The user can then use the securing members to fasten the ice pack holder 11 to the user's body part. In the illustrated embodiment, the ice pack holder 11 comprises an upper strap 16, a lower strap 17, and a center strap 18, wherein the upper, lower, and center straps 16, 17, 18 are encircled around the user's arm and are secured to a desired tightness via fasteners thereon, such as hook and loop fastening material.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An ice pack holder, comprising:
a frame defining an open central area, wherein a netting extends across the frame and over the open central area, the netting adapted to secure an ice pack thereunder;
one or more securing members extending from the frame and adapted to encircle a body part of a user so as to hold the frame in a desired position on the body of the user.

2. The ice pack holder of claim 1, wherein the frame is composed of a fabric material.

3. The ice pack holder of claim 1, wherein the netting is composed of an elastic material.

4. The ice pack holder of claim 1, wherein the one or more securing members comprise one or more pairs of straps extending from opposing sides of the frame, wherein each of the one or more pairs of straps can be removably fastened together around a body part of the user.

5. The ice pack holder of claim 1, wherein the one or more securing members are adjustable.

6. The ice pack holder of claim 1, wherein the one or more securing members comprises a D-ring disposed on a first side of the frame, and a strap extending from a second side of the frame, wherein the strap is adapted to encircle a body part of the user and extend through the D-ring in order to secure the frame in position.

7. An ice pack holder, comprising:
a frame defining an open central area, wherein a netting extends across the frame and over the open central area, the netting adapted to secure an ice pack thereunder;
a pair of upper straps extending from opposing sides of an upper end of the frame, wherein the pair of upper straps are adapted to be removably secured together around a body part of a user.

8. The ice pack holder of claim 7, further comprising a pair of lower straps extending from opposing sides of a lower end of the frame, wherein the pair of lower straps are adapted to be removably secured together around a body part of the user.

9. The ice pack holder of claim 7, wherein the pair of upper straps comprises fasteners thereon for removably secure the pair of upper straps to one another.

10. The ice pack holder of claim 9, wherein the fasteners comprise hook and loop fastening material.
